SEQUENCE LISTING (2 sequences)

SEQ ID NO: 1 - protein, 21 aa, Artificial Sequence
  Synthetic Construct
KRKKKGKGLGKKRDPRLRKYK

SEQ ID NO: 2 - protein, 21 aa, Artificial Sequence
  Synthetic Construct
KRKKKGKGLGKKRDPKLRKYK

(19) United States
(12) Patent Application Publication
(10) Pub. No.: US 2017/0328886 A1
(43) Pub. Date: Nov. 16, 2017

(54) SYSTEMS AND METHODS FOR DETERMINING THERAPEUTIC UPTAKE AND DOSING

(71) Applicants: ProteoThera, Inc., Cambridge, MA (US); Massachusetts Institute of Technology, Cambridge, MA (US)

(72) Inventors: Parth PATWARI, Cambridge, MA (US); Paul H. LIEBESNY, Cambridge, MA (US); Alan J. GRODZINSKY, Cambridge, MA (US)

(21) Appl. No.: 15/594,131

(22) Filed: May 12, 2017

Related U.S. Application Data
(60) Provisional application No. 62/336,216, filed on May 13, 2016.

(51) Int. Cl. G01N 33/50; C07K 14/47; G01N 33/60

(52) U.S. Cl. CPC G01N 33/502; G01N 33/5044; C07K 14/47; G01N 33/60; C07K 2319/00; G01N 2800/52

(57) ABSTRACT

Tools for characterizing uptake of therapeutic compounds by target tissue are disclosed along with methods for determining dosing regimen from the uptake parameters. Uptake parameters considered include partition coefficient, diffusivity, and equilibrium uptake ratio. Systems for determining partition coefficient and diffusivity in rapid uptake combinations of compounds and tissue are also reported.

### TECHNICAL FIELD

Systems and methods of the invention relate to measuring uptake of proteins in living tissue and in determining dosing and delivery schedules. In particular, this disclosure relates to uptake of heparin-binding domain fusion proteins into proteoglycan expressing tissue.

### BACKGROUND

Targeted delivery of therapeutic agents to specific areas or tissue of a body presents a challenge to modern medicine. Many promising therapeutics may provide great benefit when localized at a desired concentration but can have unwanted and perhaps dangerous effects in off target tissues or in systemic distribution. One example of a challenging treatment technique is cartilage via intra-articular injection. Therapeutics that may show promise in ex-vivo, laboratory cartilage treatment may not be practical in in-vivo treatment due to the rapid clearance of small and large molecules from the joint via sub-synovial capillaries and lymphatics. Recent developments may address this challenge by providing for selective delivery of recombinant therapeutic proteins or small molecules to cells or tissues that express proteoglycans, (e.g., cartilage, brain and spinal cord tissue, skin and subcutaneous tissue) using heparin-binding peptide linked therapeutics that exhibit significantly increased uptake and retention when compared to the therapeutic alone. See, for example, Lee, et al., U.S. patent application Ser. No. 14/409,270, incorporated herein by reference.

Because the linked therapeutics may still pose a systemic risk, accurately characterizing the uptake and retention of targeted therapeutics is essential to determining an efficient dosing regimen that minimizes systemic exposure while still providing the desired therapeutic effect at the target site. Determining the correct dosing regimen for targeted therapeutics delivered to tissues presents a further challenge because it is generally not feasible to measure the concentration of the therapeutic within the targeted tissue in patients. The concentration profile over time within a targeted tissue can be measured in an animal. However, there are no methods available for scaling such information from the animal to determine the concentration profile over time in the targeted tissue in humans, thus allowing determination of the correct dose and dosing interval.

### SUMMARY

Systems and methods of the invention provide the tools for characterizing tissue uptake of therapeutic proteins or small molecules and for determining appropriate dosing regimens based on such characterizations. The invention identifies key parameters for uptake modeling and dosing determination including equilibrium uptake ratio, partition coefficient, and diffusivity. Systems and methods of the invention further provide means for testing and quantifying these parameters including new methods for analyzing the kinetics in rapid uptake situations such as between heparin-binding (HB) fusion proteins and proteoglycan rich tissue. By characterizing uptake in both human and animal tissues for a given compound, systems and methods of the invention also provide means for scaling and comparing the concentration profile over time within a targeted tissue across species. Because methods exist for measuring the concentration profile of compounds in targeted tissue in animals but not easily in humans, the scaling provided by the invention allows for better understanding of concentration profiles in humans.

The identified uptake parameters may be determined using radiolabeled therapeutics that may be exposed to a target tissue sample (e.g., cartilage) from a human or other species. By measuring radioactivity in the tissue after exposure and comparing it to residual radioactivity of the unretained radiolabeled therapeutic, the equilibrium uptake may be measured. Similarly, diffusivity and partition coefficient for a given therapeutic and tissue may be determined from diffusive flux by measuring radioactivity of a buffer containing the radiolabeled therapeutic both upstream and downstream of a target tissue sample. Alternatively, radioactivity may by monitored during exposure of the target tissue sample to the radiolabeled therapeutic and the measured signal decay may be used to determine the diffusivity and partition coefficient as on insight of the invention is that radioactivity in the radiolabeled therapeutic bath decreases as the radiolabeled therapeutics leave the fluid phase and enter the target tissue.

The diffusivity, equilibrium uptake, and partition coefficient for a therapeutic at various concentrations, may be used to model the amount of the therapeutic that will enter a target tissue and the speed with which it will do so in a clinical setting. From this information, along with knowledge of what amount of the therapeutic compound provides a desired benefit and what amount may risk side effects (quantities that may be experimentally determined using known techniques), a dosing regimen including dosing amounts, concentrations and administration schedules and methods can be determined to meet the therapeutic goals.

Aspects of the invention include methods for determining a clinical dosing regimen for a therapeutic protein. Steps of the method may include measuring an equilibrium uptake ratio for the therapeutic protein into a tissue sample and determining a partition coefficient for the therapeutic protein and the tissue sample. Steps of the method may include measuring the uptake ratio over time (i.e., a dynamic uptake ratio) to determine the partition coefficient and the ratio of the binding site density to the binding dissociation constant. Further steps of the method can include determining diffusivity of the therapeutic protein into the tissue sample and creating a dosing regimen for administration of the therapeutic to a target tissue of a patient based on the measured equilibrium uptake ratio and determined partition coefficient and diffusivity, wherein the target tissue and the tissue sample are of a same tissue type.

In various embodiments, the dosing regimen may comprise an administration amount or concentration, an administration schedule, or a delivery composition or method (e.g., intraarticular injection). Steps of the method may include determining a size of the target tissue in the patient and creating a dosing regimen based on the size of the target tissue in the patient. In certain embodiments, the target tissue, such as cartilage in a joint, may have been damaged and steps of the method may include assessing that damage and creating the dosing regimen based on the extent of the damage to the target tissue.

In certain embodiments, steps of the method may include determining a weight or body composition of the patient and creating the dosing regimen based on the weight or body composition. The target tissue and the tissue sample may be cartilage. According to certain systems and methods of the invention, the therapeutic protein may be a fusion protein comprising a heparin binding (HB) peptide. The HB peptide may comprise a substitution at the cysteine of a naturally occurring HB peptide or may have a sequence comprising KRKKKGKGLGKKRDPRLRKYK (SEQ ID NO:1) or KRKKKGKGLGKKRDPKLRKYK (SEQ ID NO:2). The fusion protein may include an active agent selected from the group consisting of a chemical entity to be administered to a subject to treat a condition and a biological product to be administered to a subject to treat a condition. The fusion protein may further comprise a linker configured to couple the HB peptide to the active agent and the linker, may comprise the sequence GGG in certain embodiments.

In some embodiments, steps of the method may include measuring the equilibrium uptake ratio by obtaining a radiolabeled version of the therapeutic protein; incubating the tissue sample in a bath with the radiolabeled version of the therapeutic protein; removing the tissue sample from the bath; and measuring radioactivity in the removed tissue sample and the bath after the tissue sample is removed. The radiolabel can include a radioisotope of iodine such as iodine 125 (¹²⁵I). Determining the partition coefficient and determining the diffusivity can include the steps of obtaining a radiolabeled version of the therapeutic protein; incubating the tissue sample in a bath with the radiolabeled version of the therapeutic protein; monitoring radioactivity of the bath during the incubating step; determining a signal decay for the radioactivity of the bath during the incubating step; and fitting the signal decay to a model to determine a product of the partition coefficient multiplied by the diffusivity.

In certain aspects, the invention may include a system for determining diffusivity of a compound into a tissue sample. The system can comprise a bath containing a radiolabeled compound, a tissue sample located in the bath, and a radiation detector positioned to detect radiation in the bath. The radiation detector can be a radio-chromatography detector.

### BRIEF DESCRIPTION OF THE DRAWINGS

FIG. 1 shows a system for measuring dynamic uptake of a therapeutic agent by a target tissue.

FIG. 2 shows a system for measuring equilibrium uptake of a therapeutic agent by a target tissue.

FIG. 3 shows a system for measuring rapid dynamic uptake of a therapeutic agent by a target tissue.

FIGS. 4A, 4B, and 4C are graphs that show the uptake ratio of iodine 125 (¹²⁵I) radiolabeled heparin-binding peptide (HB) linked insulin-like growth factor 1 (IGF-1) in human and bovine cartilage.

FIG. 5 shows diffusive transport of ¹²⁵I-IGF-1 across a 275 μm thick human cartilage explant.

FIGS. 6A, 6B, and 6C are graphs that show dynamic uptake of ¹²⁵I-HB-IGF-1 and ¹²⁵I-IGF-1 into human cartilage.

FIG. 7 shows a modelling of the state of a solute after delivery into tissue.

### DETAILED DESCRIPTION

The invention provides systems and methods for characterizing the uptake of therapeutic agents by target tissue and using that information to determine a dosing regimen for treating the target tissue in a patient with the therapeutic agent. Uptake is characterized by determining the diffusivity, partition coefficient, and equilibrium uptake ratio for a given therapeutic agent by a tissue sample corresponding to the target tissue to be treated. The determined uptake parameters may be combined with therapeutic dose and side effect data, patient history and parameters (e.g., weight, body composition, age, or damage in the target tissue) to create a dosing regimen or treatment plan for the patient. The dosing regimen may include a preferred formulation or concentration and administration route (e.g., suspension for intraarticular into cartilage or oral administration or a cream for topical application). Dosing regimens may also include an administration amount based on the therapeutic index or ratio of the therapeutic as measured experimentally. An administration schedule with multiple, separate treatments may also be indicated and created.

Parameters useful in characterizing therapeutic compound uptake may include equilibrium uptake ratio for the therapeutic protein into a tissue, a partition coefficient for the therapeutic protein and the tissue, and diffusivity of the therapeutic protein into the tissue. These parameters may be determined experimentally using methods and systems of the invention.

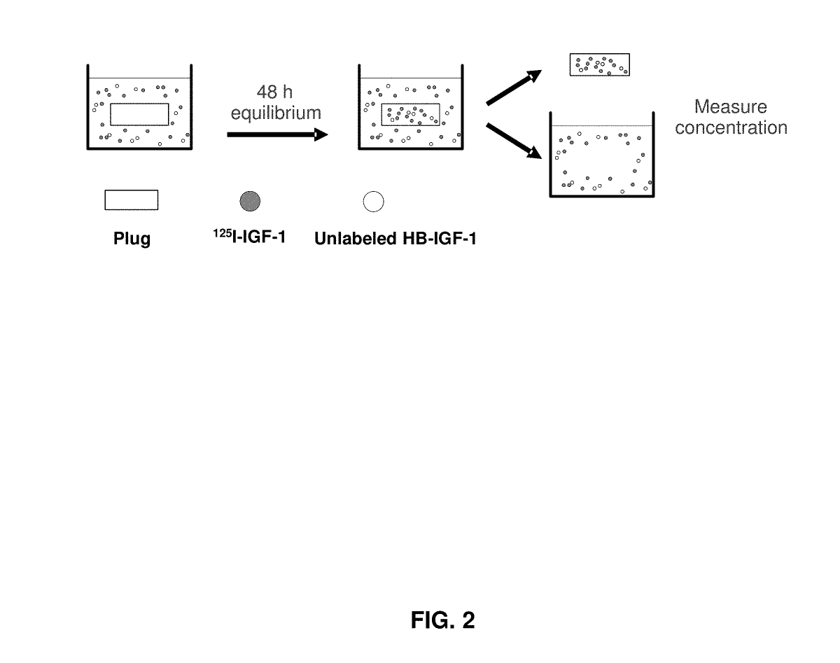

The equilibrium uptake ratio may be determined through any known method. In a preferred method, a radiolabeled version of the therapeutic protein is obtained and added to a bath at a known concentration along with a tissue sample corresponding to the tissue to be treated. The radiolabel can include any a radioisotope such as a radioisotope of, for example iodine (e.g., iodine 125 (¹²⁵I), iodine 123, iodine 124, or iodine 131). The tissue can be incubated in the bath until equilibrium is reached, after which the tissue sample may be removed and the radiation level of the removed tissue and the bath can be measured and compared (for example, using any known radiation measurement device). The uptake ratio R_{U} may be defined as the concentration of the bound and free radiolabeled therapeutic compound in the tissue per tissue weight (e.g., CPM/mg) normalized to the concentration of the radiolabeled therapeutic compound in the equilibrium bath (e.g., CPM/mL). An exemplary application of this method is depicted in FIG. 2. The uptake ratio may be related to the partition coefficient (Kₚₐᵣₜ) as follows:

$${R}_{U} = \frac{{C}_{B} + {C}_{F}}{{C}_{bath}} = {K}_{part} ( 1 + \frac{n}{{K}_{d} + {C}_{F}} ) = {K}_{part} ( 1 + \frac{n}{{K}_{d} + {K}_{part} {C}_{bath}} )$$

where n is the binding site density, K_{d} is the equilibrium binding constant, and C_{bath} is the concentration of the therapeutic in the bath. Where C_{bath} is much greater than the binding site density in the tissue, the partition coefficient may be approximated by the uptake ratio (R_{U}=Kₚₐᵣₜ). Accordingly, methods of the invention may include determining the binding site density of the tissue and measuring the uptake ratio at a sufficiently high concentration such that the partition coefficient may be determined.

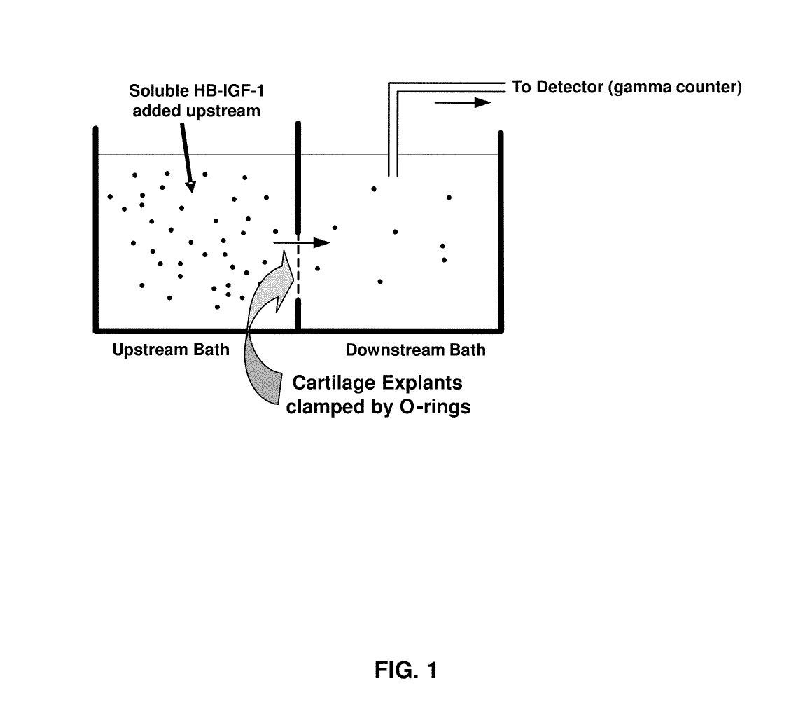

Diffusivity may be determined by any known method. In preferred embodiments, the diffusive flux of the therapeutic compound through a target tissue sample may be continuously monitored in real-time using a radiation detector such as the Radiomatic Radio-Chromatography Detector available from Canberra, Inc. (Meriden, Connecticut) or the Radiomatic A-500 Flo-one beta radio chromatography detector available from Perkin Elmer (Waltham, Mass.). Diffusivity may be measured using a diffusion chamber as shown in FIG. 1. The diffusion chamber is divided into two chambers by a tissue sample clamped by O-rings. A soluble radiolabeled therapeutic compound is added to the upper chamber (upstream bath) and the diffusion of the radiolabeled therapeutic compound from the upstream chamber or bath to the downstream chamber or bath is measured using the radiation detector and normalized to the initial upstream radiation level. This measured diffusive flux (Γ) can be related to steady-state diffusivity, Dₛₛ as follows:

$$Γ = Φ {K}_{part} {D}_{ss} \frac{ {C}_{U} - {C}_{D}}{δ} ≅ Φ {K}_{part} {D}_{ss} \frac{{C}_{U}}{δ}$$

where Φ is the tissue porosity which may be determined experimentally using known techniques or can be assumed to be a value such as 1. C_{U} and C_{D} are the upstream and downstream concentrations of the radiolabeled therapeutic compound, respectively. Retention of the compound in the tissue may also be measured and determined using this apparatus.

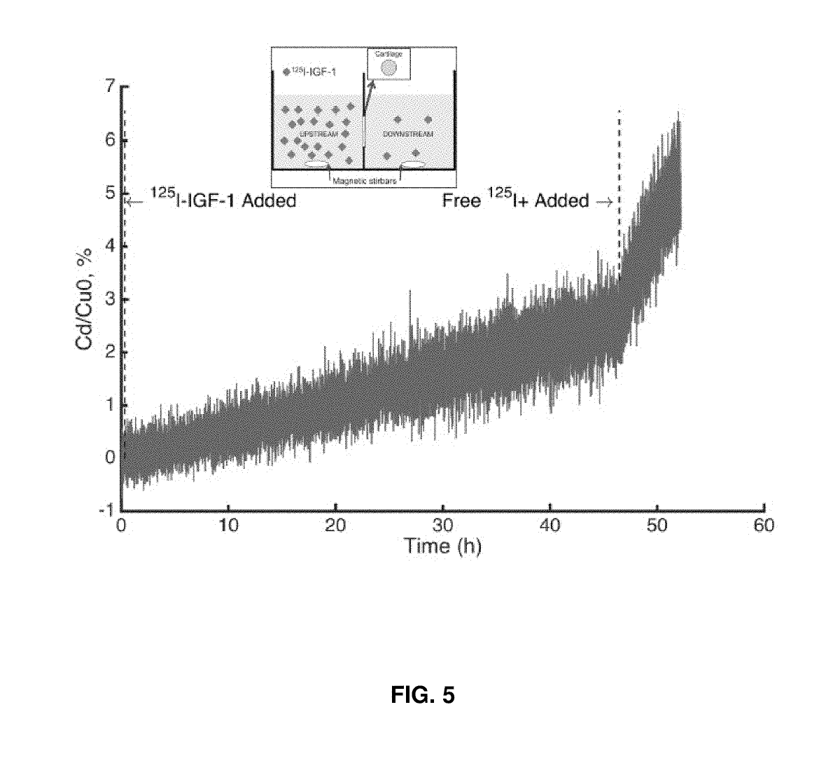

The normalized linear slope of the continuous flux can be related to the time derivative of the above diffusive flux equation as follows:

$$\frac{\partial }{\partial t} ( \frac{{C}_{D}}{{C}_{U}} ) = \frac{Γ A}{{V}_{D} {C}_{U}} ≅ \frac{Φ {K}_{part} {D}_{ss} A}{δ {V}_{D}}$$

where A is the tissue surface area exposed to the upstream compartment, and V_{D} is the volume of the downstream compartment, including any tubing leading to the detector. The slope of the ratio of C_{D} to C_{U} as measured over time is determined (see FIG. 5 for an example). That slope is fit to the equation immediately above to determine the product of the partition coefficient and the steady-state diffusivity (or diffusivity) for the therapeutic compound and the target tissue.

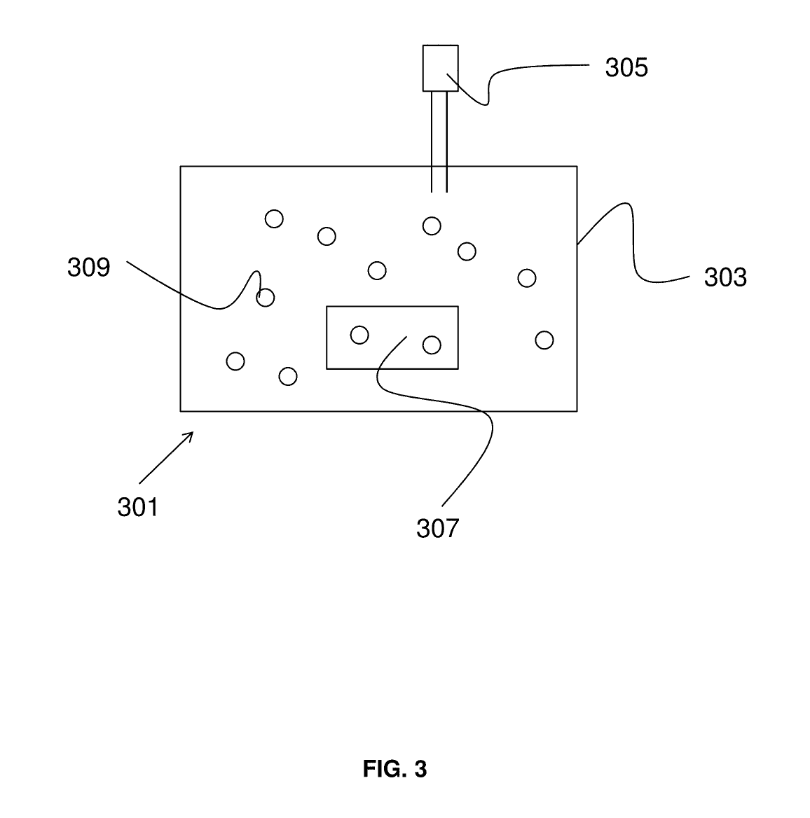

With certain therapeutic compounds and target tissue types (e.g., HB peptide linked therapeutic compounds and cartilage), uptake and retention may be so rapid and great that no downstream signal is given using the above apparatus and method. To determine diffusivity in such situations, systems of the invention such as exemplarily depicted in FIG. 3 may be used. FIG. 3 shows a system 301 for determining diffusivity of a compound 309 into a tissue sample 307. The system comprises a bath 303 containing a radiolabeled compound 309 and a tissue sample 307. A radiation detector 305 such as the types described above, is positioned to detect radiation in the bath.

One insight of the present invention is that radioactivity of the bath can be continuously monitored over time and that, in rapid uptake and high retention situations, the radioactivity decreases proportionately to the amount of radiolabeled compound exiting the liquid phase in the bath and entering the tissue. Accordingly, the decay signal from the bath can approximate the steady state diffusivity and partition coefficient of the tissue and compound. To do so, the signal decay may be fit to a nonlinear, finite difference Crank-Nicholson solution of a governing diffusion reaction model. Doing so, as shown in Example 1 below, can be used to determine a value for the product of the partition coefficient and the diffusivity for the therapeutic compound and target tissue. Where the partition coefficient cannot be determined as above using a sufficiently high bath concentration of the therapeutic compound, it may be determined by using a Stokes-Einstein equation to relate diffusivity to that of a known compound based on hydrodynamic radius and molecular weight of the known compound and the experimental compound (see Example 1 below for an application of this method).

In certain embodiments, Kpart, Dss, and n/Kd may be determined in vitro in human post-mortem donor tissue, and the clearance rate of the therapeutic compound in the synovial space may be known or measured in humans. The model may be then be used to determine a table or formula for the correct adjustments to the dose and/or dosing regimen based on the measured thickness of cartilage in a given patient, for a therapy that is delivered locally to the synovial space (e.g. by intra-articular injection).

Once the equilibrium uptake ratio, the partition coefficient, and/or the diffusivity of the therapeutic compound is determined for the target tissue, those parameters may be used to create a dosing regimen for administering the therapeutic agent or compound to the target tissue in a patient in need of treatment. After a clinical dose and dosing regimen has been identified, the dosing may be adjusted specifically for each patient based on the spatial characteristics of that patient's tissues using, for example, the following model.

Given the experimental determination of the diffusivity (D), partition coefficient (Kpart), and binding site density to equilibrium dissociation constant (n/Kd) parameters for a solute (e.g., therapeutic compound) using tissue of thickness L1, methods of the invention may be used to model the spatial and temporal uptake and transport of that molecule into an intact tissue of thickness L2. To do so one may use a numerical solution of a nonlinear diffusion-reaction model by:

$$\frac{\partial {\overline{C}}_{F}}{\partial t} ( 1 + \frac{{nK}_{d}}{{( {K}_{d} + {\overline{C}}_{F} )}^{2}} ) = D \frac{{\partial }^{2} {\overline{C}}_{F}}{\partial {x}^{2}}$$

where C̅_{F} is the free solute in the tissue, t is time, and x is the position in the tissue, where x=0 is defined as the surface of the tissue. To the above, the following boundary conditions may be applied:
i) C_{F}(X =0⁺) =KₚₐᵣₜC_{S}, where Cₛ is the known time-dependent concentration profile of the solute in the synovial space, and x=0 is the surface of the tissue.
ii) An impermeable wall no-flux boundary at x=L₂.

Boundary condition (ii) applies, for example, to the inner surface of cartilage abutting subchondral bone, assuming that this interface is non-penetrable to flow of solutes. From solution of the diffusion-reaction equation, one may obtain the 1D concentration profile of the solute in time and space in the tissue of thickness L₂, given by C̅_{F}(x, t). As the synovial fluid turns over, and the mean concentration of solute in the synovial space, C_{S}, reduces below the concentration in the tissue towards zero; a similar theoretical model may be applied to obtain the diffusive loss of the solute back into the synovial space, where Cₛ is now defined as:

$$\frac{\partial {C}_{s}}{\partial t} = D \frac{{\partial }^{2} {C}_{s}}{\partial {x}^{2}} - {kC}_{s}$$

with k denoting the clearance rate of Cₛ from the synovial space, and the boundary conditions being

C_{S}(x=0⁻)=C̅_{F}(x=0⁺, t) i)

C_{S}(x=−∞)=0 ii)

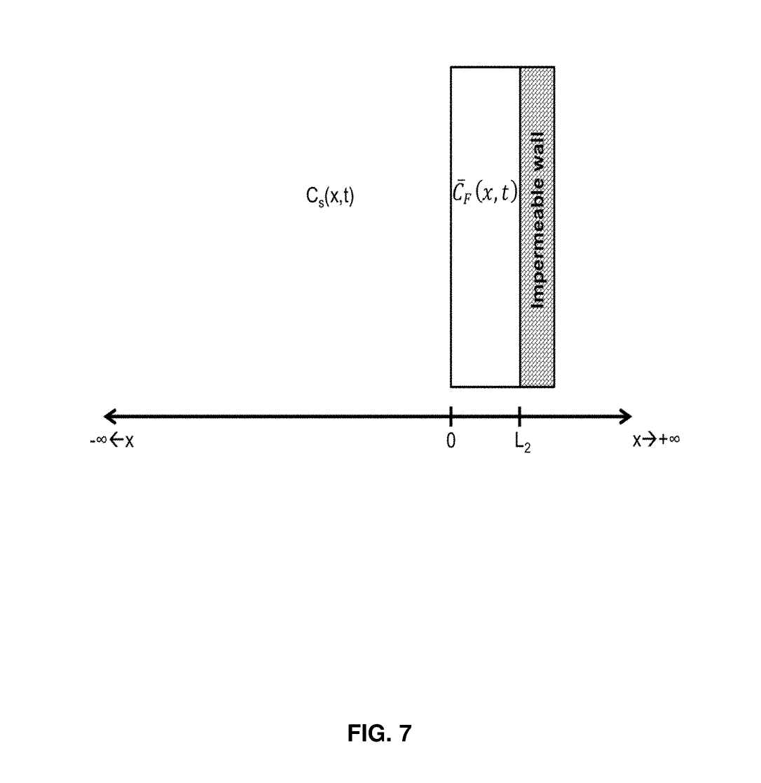

From this, the residence time of the solute in the tissue, and the overall clearance of it out of the joint space may be obtained. Superposing the two phases of the process provides a comprehensive picture of the state of the solute after delivery as shown in FIG. 7.

In certain embodiments, the diffusion-reaction model may further encompass additional adjustments for convection due to loading, and “electrodiffusion” of solutes within the tissue.

As noted above, determining the correct dosing regimen for targeted therapeutics delivered to tissues is challenging because it is generally not feasible to measure the concentration of the therapeutic within the targeted tissue in patients. However, the concentration profile over time within a targeted tissue can be measured in an animal. Without methods for scaling or comparing the relative uptake in the animal's tissue to that of a human, the benefits of animal data remain limited. In certain embodiments, of the invention systems and methods described herein may be used with both animal and human tissue samples to describe and/or quantify a relationship between their relative uptakes for a given therapeutic. Once this relationship is characterized, methods of the invention may include analyzing concentration profile data over time within a targeted tissue in an animal to provide more detailed concentration profile data for human tissue. These methods provide for scaling such information from the animal to humans (based on the differences in cartilage thickness between species) to determine the concentration profile over time in the targeted tissue in humans, thus allowing determination of the correct dose and dosing interval.

The dosing regimen may comprise an administration amount or concentration, an administration schedule, or a delivery composition or method (e.g., intraarticular injection).

Additional considerations in determining a dosing regimen may include determining a size of the target tissue in the patient (e.g., a volume of cartilage to be treated in a damaged joint), the extent of damage to a tissue in need of treatment (e.g., the extent of cartilage degradation in a patient's joint), or the weight or body composition (e.g., percentages of muscle, fat, or fluids) of the patient.

Dosing information may be determined experimentally or may be obtained from a source such as the U.S. Food and Drug Administration. Experimental determination may include animal or human studies of the therapeutic compound and determination of a therapeutic index based on the ratio of a lethal or toxic dose to a minimum effective dose.

Determining a dosing regimen based on the uptake parameters may include determining a pharmaceutically acceptable formulation for delivery of the therapeutic compound. Such a pharmaceutically acceptable formulation may include a pharmaceutically acceptable carrier(s) or excipient(s), solvents, dispersion media, coatings, antibacterial and antifungal agents, isotonic and absorption delaying agents, and the like, that are physiologically compatible. For example, the carrier can be suitable for intra-articular injection. Excipients include pharmaceutically acceptable stabilizers. The present invention pertains to any pharmaceutically acceptable formulations, including synthetic or natural polymers in the form of macromolecular complexes, nanocapsules, microspheres, or beads, and lipid-based formulations including oil-in-water emulsions, micelles, mixed micelles, synthetic membrane vesicles, and gels such as hyaluronic gels.

In some embodiments, a composition comprising an analyzed therapeutic compound as disclosed herein can be formulated in any suitable means, e.g., as a sterile injectable solution, e.g., which can be prepared by incorporating an analyzed therapeutic compound in the required amount of the appropriate solvent with various of the other ingredients, as desired. In some embodiments, a composition comprising an analyzed therapeutic compound as disclosed herein can be formulated in a hydrogel, for example, but not limited to a hydrogel comprising self-assembling peptides is RADA-16 (also known as PURAMATRIX®), KLD-12, and KLDL-12.

A pharmacological formulation of a composition comprising an analyzed therapeutic compound as disclosed herein can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those presented in U.S. Pat. Nos. 5,225,182; 5,169,383; 5,167,616; 4,959,217; 4,925,678; 4,487,603; 4,486,194; 4,447,233; 4,447, 224; 4,439,196 and 4,475,196. Other such implants, delivery systems, and modules are well known to those skilled in the art.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, e.g., parabens, chlorobutanol, phenol and sorbic acid. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

In another embodiment, a composition comprising an analyzed therapeutic compound as disclosed herein can comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used. Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidyleholine, dioleoylphosphatidylcholine, dipalmitoylphosphatidylglycerol, and dioleoylphosphatidylglycerol.

In preparing lipid-based vesicles containing an analyzed therapeutic compound, such variables as the efficiency of active compound encapsulation, labiality of the active compound, homogeneity and size of the resulting population of vesicles, active compound-to-lipid ratio, permeability, instability of the preparation, and pharmaceutical acceptability of the formulation should be considered.

In another embodiment, an analyzed therapeutic compound can be delivered in a vesicle, in particular a liposome (see Langer (1990) Science 249:1527-1533). In yet another embodiment, an analyzed therapeutic compound can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding an analyzed therapeutic compound, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Prior to introduction, a composition comprising an analyzed therapeutic compound as disclosed herein can be sterilized, by any of the numerous available techniques of the art, such as with gamma radiation or electron beam sterilization.

In another embodiment of the invention, a composition comprising an analyzed therapeutic compound or variant thereof as disclosed herein, can be administered and/or formulated in conjunction (e.g., in combination) with any other therapeutic agent. For purpose of administration, an analyzed therapeutic compound as disclosed herein is preferably formulated as a pharmaceutical composition. Pharmaceutical compositions of the present invention comprise a compound of this invention and a pharmaceutically acceptable carrier, wherein the compound is present in the composition in an amount which is effective to treat the condition of interest. Appropriate concentrations and dosages can be readily determined by one skilled in the art. Pharmaceutically acceptable carriers are familiar to those skilled in the art. For compositions formulated as liquid solutions, acceptable carriers include saline and sterile water, and may optionally include antioxidants, buffers, bacteriostats and other common additives. The compositions can also be formulated as pills, capsules, granules, or tablets which contain, in addition to a compound of this invention, diluents, dispersing and surface active agents, binders, and lubricants. One skilled in this art may further formulate the compounds of this invention in an appropriate manner, and in accordance with accepted practices, such as those disclosed in Remington's Pharmaceutical Sciences, Gennaro, Ed., Mack Publishing Co., Easton, Pa. 1990.

The compositions of the present invention can be in any form. These forms include, but are not limited to, solutions, suspensions, dispersions, ointments (including oral ointments), creams, pastes, gels, powders (including tooth powders), toothpastes, lozenges, salve, chewing gum, mouth sprays, pastilles, sachets, mouthwashes, aerosols, tablets, capsules, transdermal patches, that comprise one or more resolvins and/or protectins or their analogues of the invention.

Formulations of a composition comprising an analyzed therapeutic compound as disclosed herein can be prepared by a number or means known to persons skilled in the art. In some embodiments the formulations can be prepared for administration as an aerosol formulation, e.g., by combining (i) an analyzed therapeutic compound as disclosed herein in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

In certain embodiments, a composition comprising an analyzed therapeutic compound, which is a nucleic acid agent or polypeptide agent can be administered to a subject as a pharmaceutical composition with a pharmaceutically acceptable carrier. In certain embodiments, these pharmaceutical compositions optionally further comprise one or more additional therapeutic agents. In certain embodiments, the additional therapeutic agent or agents are autoimmune disease or drugs, such as immune suppressants and the like. Of course, such therapeutic agents are which are known to those of ordinary skill in the art can readily be substituted as this list should not be considered exhaustive or limiting.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: water soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs.

In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some instances, a composition comprising an analyzed therapeutic compound as disclosed herein can be in a formulation suitable for rectal or vaginal administration, for example as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore release the active compound. Suitable carriers and formulations for such administration are known in the art.

Dosage forms for the topical or transdermal administration of an analyzed therapeutic compound, e.g., for muscular administration include powders, sprays, ointments, pastes, creams, lotions, gels, solutions, patches and inhalants. An analyzed therapeutic compound as disclosed herein may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an analyzed therapeutic compound to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, an analyzed therapeutic compound can be isolated and/or purified or substantially purified by one or more purification methods described herein or known by those skilled in the art. Generally, the purities are at least 90%, in particular 95% and often greater than 99%. In certain embodiments, the naturally occurring compound is excluded from the general description of the broader genus.

In some embodiments, the composition comprises at least one an analyzed therapeutic compound in combination with a pharmaceutically acceptable carrier. Some examples of materials which can serve as pharmaceutically acceptable carriers include, without limitation: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, a composition comprising an analyzed therapeutic compound as disclosed herein can contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention.

These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of patients without undue toxicity, irritation, allergic response, and the like, commensurate with a reasonable benefit/risk ratio, and effective for their intended use. These compounds include the zwitterionic forms, where possible, of r compounds of the invention.

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “prodrug” refers to compounds or agents that are rapidly transformed in vivo to yield the active an analyzed therapeutic compound, e.g., a biologically active or functional active an analyzed therapeutic compound which encodes a functionally active therapeutic peptide or protein. In some embodiments, an analyzed therapeutic compound prodrugs can be activated by hydrolysis in blood, e.g., via cleavage of a precursor therapeutic protein into an active therapeutic protein, similar to how insulin is activated from its proprotein into an active insulin protein. A thorough discussion is provided in T. Higachi and V. Stella, “Pro-drugs as Novel Delivery Systems,” Vol. 14 of the A.C.S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. The prodrug may be designed to alter the metabolic stability or the transport characteristics of an analyzed therapeutic compound, to mask side effects or toxicity, or to alter other characteristics or properties of an analyzed therapeutic compound. By virtue of knowledge of pharmacodynamic processes and drug metabolism or post-translational protein processing of an analyzed therapeutic compound in vivo, once a pharmaceutically active compound is identified, those of skill in the pharmaceutical art generally can design an analyzed therapeutic compound prodrugs which can be activated in vivo to increase levels of the therapeutic protein present in an analyzed therapeutic compound in the subject (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

Target tissues may include intact cells, blood, blood preparations such as plasma and serum, bones, joints, cartilage, neuronal tissue (brain, spinal cord and neurons), muscles, smooth muscles, and organs. Compounds may include any known therapeutic compound. Compounds may comprise a heparin binding motif such as KRKKKGKGLGKKRDPRLRKYK (SEQ ID NO:1) or KRKKKGKGLGKKRDPKLRKYK (SEQ ID NO:2) or any other such as described in Lee, et al. Proteins created by fusion with a peptide derived from a heparin-binding domain do not bind only to heparin or heparin sulfate. The peptide may also be characterized by binding to other glycosaminoglycans such as chondroitin sulfate. HB-IGF-1 is known to bind to both heparin sulfate and chondriotin sulfate, where binding to chondroitin sulfated glycosaminoglycans is of primary importance for binding of the HB-IGF-1 to cartilage. See Miller, Grodzinsky, Patwari et al., Arthritis and Rheum 2010, incorporated herein by reference.

The fusion protein may include an active agent selected from the group consisting of a chemical entity to be administered to a subject to treat a condition and a biological product to be administered to a subject to treat a condition.

Active agents may include therapeutic proteins or small molecules such as: Neurotrophic factors; Neurothrophins; nerve growth factor (NGF); brain-derived neurotrophic factor (BDNF); neurotrophin-3 (NT-3); neurotrophin-4 (NT-4); Ciliary neurotrophic factor (CNTF); mesencephalic astrocyte-derived neurotrophic factor (MANF); conserved dopamine neurotrophic factor (CDNF); Glial cell-line derived neurotrophic factor family ligands; glial cell line-derived neurotrophic factor (GDNF); neurturin (NRTN); artemin (ARTN); or persephin (PSPN); Neuropoietic cytokines; interleukin-6; interleukin-11; interleukin 27; leukaemia inhibitory factor; ciliary neurotrophic factor; cardiotrophin 1; neuropoietin; cardiotrophin-like cytokine; Fibroblast Growth Factor 2; Anti-inflammatory cytokines; interleukin-4; interleukin-10; Neuroprotection agents; Neuregulin-1; Vascular endothelial growth factor (VEGF); Cerebrolysin® (FPF 1070), Etanercept (Enbrel®, soluble recombinant TNF receptor 2 fused to the Fc component of human immunoglobulin G1); Growth differentiation factor 11 (GDF11); Stromal cell-derived factor 1 (SDF-1); Myostatin (growth differentiation factor 8 (GDF8)); Parathyroid hormone (PTH); Parathyroid hormone related peptide (PTHrP); Interleukin 1 receptor antagonist (IL-1RA); Fibroblast growth factor 18 (FGF-18); High-mobility group protein 2 (HMG-2, also known as High mobility group box 2 (HMGB2)); Glucocorticoid receptor; a therapeutic antibody or portion thereof, such as Remicade® (infliximab, anti-TNF-α, Janssen Biotech, Horsham, Pa.), Humira® (adalimumab, anti TNF, Abbot Labs., N. Chicago, Ill.), or an anti-nerve growth factor antibody; Fibroblast growth factor 9 (FGF 9); Hepatocyte growth factor; TGFβ-superfamily proteins such as TGFβ, TGFβ3, BMP2, or BMP7; or other therapeutic proteins; or functional portions, variants, analogs, or derivatives of any of the foregoing; or small molecule active agents.

Fusion proteins may further comprise a linker configured to couple the HB peptide to the active agent. Linkers may be a peptide such as GGG or may comprise other linkers as described in Lee, et al.

### EXAMPLES

### Materials and Methods

Cartilage explant disks 6 mm in diameter with the superficial zone intact were obtained from the femoropatellar grooves of immature bovine calves (Research 87, Boylston, Mass.) using a 6 mm biopsy punch. Specimens used for diffusive transport studies were trimmed to a final thickness of −500 μm, including the superficial zone. Fore specimens used for equilibrium uptake studies, the first 0.7 mm of tissue, including the superficial zone, was removed from the surface of full thickness plugs, and up to 3 sequential 1.4 mm slices of middle-zone tissue were taken from the remainder of the plug, depending on the harvest location. These plugs were then cored to final diameters of 3 or 4 mm using a dermal punch. The wet weights of all final-sized explants were measured. Disks were incubated in serum-free medium [low-glucose Dulbecco's Modified Eagle's Medium (DMEM; 1 g/L) (Mediatech, Inc., Manassas, Va.) supplemented with penicillin-streptomycin-amphotericin (PSA), 4-(2-hydroxyethyl)-1-piperzaineethanesulfonic acid (HEPES) (Invitrogen, Carlsbad, Calif.), proline, ascorbate, and non-essential amino acids (NEAA) (Sigma-Aldrich)] if being used the same day as harvest, or frozen in phosphate-buffered saline (PBS) with 1% PSA and Complete Protease Inhibitors (Roche Applied Science, Indianapolis, Ind.) until further use.

Cartilage explants from adult human knee joints with the superficial zone intact were harvested from the femoropatellar grooves and condyles from the knee of a 26-year-old male, a 62-year-old female, and a 32-year-old male (Modified Collins Grade 0 or 1) using a 6 mm dermal punch and scalpel. Disks 6 mm in diameter with intact superficial zone were trimmed to 300-500 μm thick for diffusive transport studies while the remaining middle and deep zone tissue was trimmed to 400-500 μm thick disks for dynamic uptake studies. Smaller pieces of tissue were obtained for equilibrium uptake studies using a scalpel. All samples were frozen in PBS with 1% PSA and Complete Protease Inhibitors until further use.

With long-term storage, ¹²⁵I radiolabel and/or small ¹²⁵I-fragments can degrade from their labeled ¹²⁵I-HB-IGF-1 or 125I-IGF-1 form. Thus, before use in transport or uptake studies, any small ¹²⁵I species that may have resulted from degradation of ¹²⁵I-HB-IGF-1 or ¹²⁵I-IGF-1 were removed by spin-filter purification. For applications using smaller transport chambers or higher concentrations of intact-labeled protein, a spin filter purification technique was employed to keep final purified solute volumes low. Briefly, stock labeled protein was diluted 1:10 in a 150 mM NaCl+25 mM HEPES and spun through a 3,000 MW cut off Centricon centrifugation filter (EMD Millipore, Billerica, Mass.). The volume spun through the filter was reserved, and the retained volume above the filter was re-diluted 1:10 in the same buffer. This method of serial diluting small ¹²⁵I-species was repeated 5-7 times (1:100,000 to 1:10,000,000 final dilution). The radioactivity of all retained flow-through volumes and the final purified retentate were counted with a gamma counter. The final ratio of intact, labeled protein to residual small ¹²⁵I-species was determined by gravity fed Sephadex G-50 gel filtration chromatography as above. For certain supplemental experiments in larger transport chambers, column purification was implemented.

To determine the equilibrium uptake of ¹²⁵I-HB-IGF-1 into cartilage, bovine and human cartilage explants were incubated in baths of 150 mM NaCl+25 mM HEPES +1% PSA+protease inhibitors (buffer) with known concentrations of ¹²⁵I-HB-IGF-1 (0.01-1 μM) and HB-IGF-1 (0-100 μM) at 37° C. until equilibrium (48 hours). Following incubation, samples were briefly rinsed, radioactivity was counted for each sample and bath separately using a gamma counter, and wet and dry weights of explants were measured. The uptake ratio was determined as the counts per minute (CPM) in the explant to that in the bath, normalized to wet weight. The same experiment was performed using ¹²⁵I-IGF-1 and IGF-1 for human cartilage at a single equilibrium bath concentration on ˜66 μM IGF-1.

Transport experiments were conducted to estimate the diffusive flux of ¹²⁵I-IGF-1 through adult cartilage using a method adapted from Garcia et al. (FIG. 5 inset). Briefly, one 6-mm diameter explant was clamped, superficial zone facing upstream, between two compartments of a polypropylene diffusion chamber with O-rings to prevent leakage. The compartments were filled with up to 2 mL of buffer+0.1% BSA, and spin-purified ¹²⁵I-IGF-1 was added to the upstream chamber at t=0. One-dimensional diffusion of ¹²⁵I-IGF-1 from the upstream chamber to the downstream chamber was monitored using a Radiomatic A-500 Flo-one beta radio chromatography detector (Perkin Elmer, Waltham, Mass.). The downstream signal was normalized to the initial upstream bath signal.

Due to the high uptake of ¹²⁵I-HB-IGF-1 into human cartilage, standard diffusive transport studies proved challenging, as large quantities of ¹²⁵I-HB-IGF-1 went into the tissue from the upstream compartment but were not released to the downstream side. As an alternative approach to determine the kinetics of ¹²⁵I-HB-IGF-1 into human cartilage, we conducted the following dynamic uptake experiment. All surfaces of the experimental setup were blocked with SuperBlock™ Blocking Buffer (ThermoFisher Scientific, Waltham, Mass.). ¹²⁵I-HB-IGF or ¹²⁵I-IGF-1 (as a control experiment) was added to a 3.5 mL bath of buffer with 0.1% BSA continuously monitored using a Radiomatic radio chromatography detector to establish a baseline radioactivity signal. Human cartilage explants, described above, were added to the bath, and the radioactivity signal continued to be monitored. As the 125I-labeled growth factor left the fluid phase of the bath and entered the solid phase of the cartilage, the radioactivity decayed until equilibrium was reached.

Dried samples were digested in Proteinase-K (Roche), and sGAG content was assessed by DMMB dye binding.

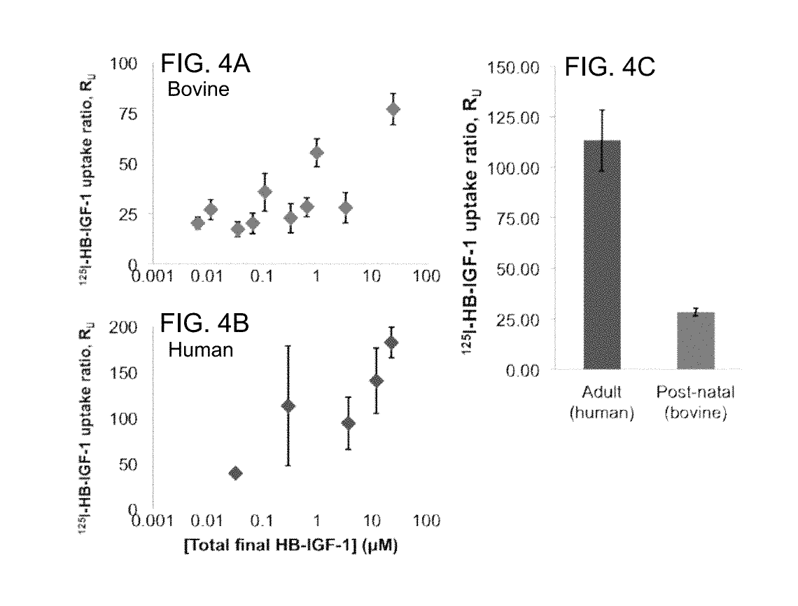

The equilibrium uptake of ¹²⁵I-HB-IGF-1 into both post-natal bovine and adult human cartilage was measured by radioactivity following 48 hours of incubation. Past studies have demonstrated this ability of HB-IGF-1 to be taken up into cartilage, but the uptake ratio was not quantified. These studies also demonstrated the ability of high salt conditions to desorb HB-IGF-1 that had entered cartilage out of the tissue, suggesting an interaction between HB-IGF-1 and cartilage that is dominated by weak electrostatic forces. The uptake ratio, R_{U}, defined as the concentration of ¹²⁵I-HB-IGF-1 in the cartilage (bound (CB) plus free (CF)) per tissue wet weight (CPM/mg) normalized to the concentration of ¹²⁵I-HB-IGF-1 in the equilibrium bath (CPM/mL):

$${R}_{U} = \frac{{C}_{B} + {C}_{F}}{{C}_{bath}} = {K}_{part} ( 1 + \frac{n}{{K}_{d} + {C}_{F}} ) = {K}_{part} ( 1 + \frac{n}{{K}_{d} + {K}_{part} {C}_{bath}} ) \qquad ( 4.1 )$$

where Kₚₐᵣₜ is the partition coefficient, n is the binding site density, K_{d} is the equilibrium binding constant, and C_{bath} is the concentration of the growth factor in the bath. Under conditions where C_{bath}>>n, a value for Kpart can be obtained, and under conditions where C_{bath}→0, the ratio of n/K_{d} can be determined. Uptake of ¹²⁵I-HB-IGF-1 quantified here was consistently high across the concentrations tested, with mean values±standard error of 114±15 for human and 28±2 for bovine (FIG. 4C). The uptake was high enough such that final equilibrium bath concentrations of the growth factor were greatly reduced compared to the initial bath concentration. Final uptake ratios were plotted as a function of these reduced final bath concentrations (7-25 μM) (FIGS. 4A and 4B). The high uptake across concentrations tested suggests that the number of available binding sites for HB-IGF-1 in cartilage was not saturated at the concentrations tested (up to 25 μM final bath concentration). This is consistent with the hypothesis of ¹²⁵I-HB-IGF-1 interacting with the charged proteoglycans in cartilage, which are bountiful.

To estimate the partitioning of IGF-1 into adult human cartilage, the uptake ratio of IGF-1 into human cartilage was determined at equilibrium bath concentrations of ˜66 μM IGF-1, using the methods described above. Based on previously published work characterizing the uptake of IGF-1 into adult bovine cartilage, the concentration of 66 μM used here is such that C_{bath}>>n, and R_{U}=Kₚₐᵣₜ. These experiments yielded an uptake ratio of R_{U}^{IGF-1}=3.15±0.52.

To determine the transport kinetics of ¹²⁵I-IGF-1 through human cartilage, the diffusive flux of ¹²⁵I-IGF-1 through a cartilage explant was continuously monitored in real-time using the Radiomatic detector connected via continuous recirculation to the downstream compartment of the diffusion chamber. After an initial lag time of ˜42 minutes following the addition of ¹²⁵I to the system, during which the protein binds to tissue, a signal was detected in the downstream compartment that rose linearly with time (FIG. 5). This flux is related to the steady-state diffusivity, Dₛₛ, by:

$$Γ = Φ {K}_{part} {D}_{ss} \frac{{C}_{U} - {C}_{D}}{δ} ≅ Φ {K}_{part} {D}_{ss} \frac{{C}_{U}}{δ} \qquad ( 4.2 )$$

where Φ is the tissue porosity (assumed to be 1 here), Kₚₐᵣₜ is the partition coefficient, and C_{U} and C_{D} are the upstream and downstream concentrations, respectively. The normalized linear slope of the continuous flux measured in FIG. 5 can be related to the time derivative of Eq. 4.2 by:

$$\frac{\partial }{\partial t} ( \frac{{C}_{D}}{{C}_{U}} ) = \frac{Γ A}{{V}_{D} {C}_{U}} ≅ \frac{Φ {K}_{part} {D}_{ss} A}{δ {V}_{D}} \qquad ( 4.3 )$$

where A is the tissue surface area exposed to the upstream compartment (0.126 cm²), and V_{D} is the volume of the downstream compartment, including the tubing of the detector (3.5 mL).

Fitting the slope generated in FIG. 5 to Eq. 4.3 yields a Kₚₐᵣₜ^{IGF-1}*D_{SS}^{IGF-1}=1.1±0.36×10⁻⁷ cm²/s.

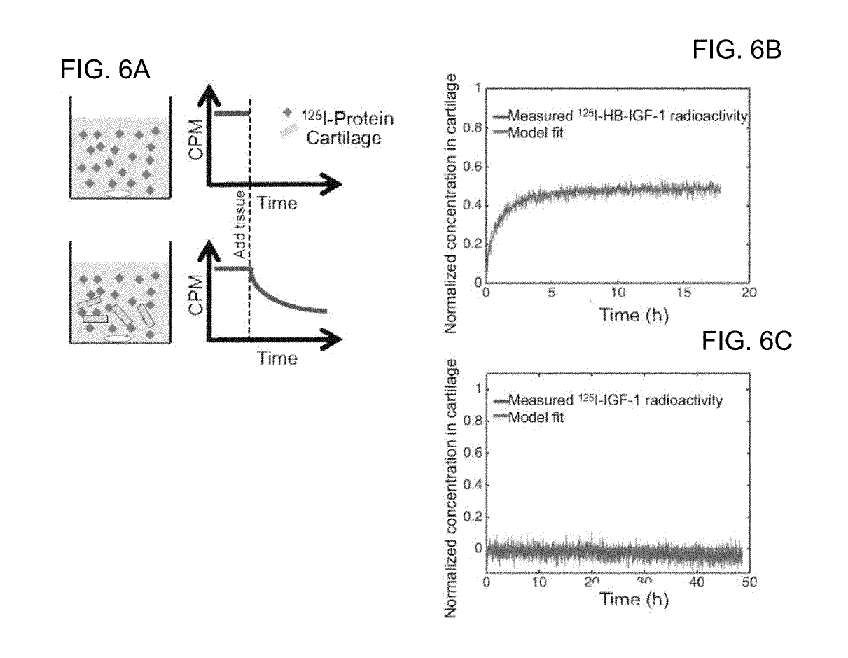

Due to the high uptake of ¹²⁵I-HB-IGF-1 into cartilage and subsequent retention of the growth factor in the tissue, traditional diffusive transport experiments yielded little to no downstream signal. Instead, we developed an alternative approach to quantify the kinetics of the interaction between ¹²⁵I-HB-IGF-1 and cartilage. Cartilage explants were added to a bath containing ¹²⁵I-HB-IGF-1 while the radioactivity in the bath was continuously monitored (FIG. 6A). As the growth factor left the fluid phase of the setup and entered the tissue, the radioactivity monitored in the bath decreased. Over n=3 donors, the system reached 90% equilibrium in an average of 2.76±0.66 hours with an average uptake ratio of ˜77. (FIG. 6B). A control experiment performed with ¹²⁵I-IGF-1 demonstrated a much lower level of uptake (R_{U}˜3.5). At this low level of uptake, any uptake dynamics were too subtle to observe with our setup, illustrating the dramatic difference induced by the addition of the HB-domain to the protein.

To extract the product of the steady-state diffusivity and the partition coefficient (as done for IGF-1 in the traditional diffusion reaction experiment in 4.3.2), Kₚₐᵣₜ^{HB-IGF-1}*D_{SS}^{HB-IGF-1}, from these experiments, the signal decay was fit to a nonlinear, finite difference, Crank-Nicholson solution of the governing diffusion reaction model shown in Eq. 4.4.

$$\frac{\partial {\overline{C}}_{F}}{\partial t} ( 1 + \frac{{nK}_{d}}{{( {K}_{d} + {\overline{C}}_{F} )}^{2}} ) = {D}_{HB \text{-} IGF} \frac{{\partial }^{2} {\overline{C}}_{F}}{\partial {x}^{2}} \qquad ( 4.4 )$$

This model fit, resulted in a Kₚₐᵣₜ^{HB-IGF-1}*D_{SS}^{HB-IGF-1}=1.32±0.13×10⁻⁶ cm²/s. To estimate a value of Kₚₐᵣₜ^{HB-IGF-1}, we took the ratio of:

$$\frac{{K}_{part}^{HB \text{-} IGF \text{-} 1} {D}_{ss}^{HB \text{-} IGF \text{-} 1}}{{K}_{part}^{IGF \text{-} 1} {D}_{ss}^{IGF \text{-} 1}} = 12.4 \pm 4.4 \qquad ( 4.5 )$$

Using the Stokes-Einstein equation to relate the diffusivity of a molecule to its hydrodynamic radius, and by extension its molecular weight, we approximated:

$$\frac{{D}_{ss}^{HB \text{-} IGF \text{-} 1}}{{D}_{ss}^{IGF \text{-} 1}} = {( \frac{M {W}^{IGF \text{-} 1}}{M {W}^{HB \text{-} IGF \text{-} 1}} )}^{\frac{1}{3}} = 0.9097 . \qquad ( 4.6 )$$

Thus, we calculated the ratio of the partition coefficients to be:

$$\frac{{K}_{part}^{HB \text{-} IGF \text{-} 1}}{{K}_{part}^{IGF \text{-} 1}} = 13.5 \pm 4.8 . \qquad ( 4.7 )$$

Utilizing a value of Kₚₐᵣₜ^{IFG-1}=1.4 for IGF-1 in adult bovine cartilage, and the value of Kₚₐᵣₜ^{IGF-1}=3.15±0.52, estimated from the ¹²⁵I-IGF-1 uptake experiment in adult human tissue above, we approximated Kₚₐᵣₜ^{HB-IGF-1} is between 19 and 43. Results from the example are shown in FIGS. 4-6.

As shown in FIGS. 4A and 4B, ¹²⁵I-HB-IGF-1 was strongly taken up into both post-natal (bovine) (FIG. 4A) and adult (human) (FIG. 4B) cartilage over a final bath concentration range from 7 nM-25 μM. Uptake ratios were determined using the counts per minute in the cartilage divided by the counts per minute in the bath, normalized by the wet weight. The uptake was shown to have a general dependence on the concentration of HB-IGF-1 (Bovine: p<0.0001, Wilcoxon test; Human: p=0.0082, ANOVA). FIG. 4C shows that across concentrations, adult human cartilage had an average uptake of 114 while post-natal bovine tissue had an average uptake of 28.

FIG. 5 shows diffusive transport of ¹²⁵I-IGF-1 across a 275 μm thick human cartilage explant (age 32). The radioactivity from emitted from ¹²⁵I-IGF-1 in the downstream chamber was continuously monitored using a Radiomatic radio chromatography detector. After establishing a buffer-only baseline for 20 minutes, 150 nM of ¹²⁵I-IGF-1 was added to the upstream compartment of the transport chamber. To confirm that the signal detected was indeed intact labeled ¹²⁵I-IGF-1, ¹²⁵I-small species collected during the spin-filter purification process were added to the upstream chamber after 46.5 hours, after which a steeper slope, indicating faster transport, was observed.

FIGS. 6A-6C show dynamic uptake of ¹²⁵I-HB-IGF and ¹²⁵I-IGF into human cartilage. Cartilage explants with the superficial zone removed (average thickness of 400-500 μm) were added to a bath containing ˜133 nM of either ¹²⁵I-HB-IGF-1 or ¹²⁵I-IGF-1, and the decrease in bath radioactivity was monitored using a Radiomatic radio chromatography detector as the labeled protein entered the cartilage (FIG. 6A). FIG. 6B shows a representative example of ¹²⁵I-HB-IGF-1 dynamic uptake. ¹²⁵I-HB-IGF-1 was dramatically and rapidly taken up into human cartilage, reaching 90% equilibrium in an average of ˜2.76 hours at an average uptake ratio ˜77. FIG. 6C shows that, at the concentration of ¹²⁵I-IGF-1 required to obtain a reliable radioactive signal (-133 nM), the uptake ratio of ¹²⁵I-IGF-1 was low at ˜3.5, and the kinetics were not observable.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for determining a clinical dosing regimen for a therapeutic protein, the method comprising:
measuring an equilibrium uptake ratio for the therapeutic protein into a tissue sample;
determining a partition coefficient for the therapeutic protein and the tissue sample;
determining diffusivity of the therapeutic protein in the tissue sample;
determining a ratio for a binding site density of the tissue sample to an equilibrium dissociation constant; and
creating a dosing regimen for administration of the therapeutic to a target tissue of a patient based on the measured equilibrium uptake ratio and determined partition coefficient and diffusivity, wherein the target tissue and the tissue sample are of a same tissue type.

2. The method of claim 1, wherein the dosing regimen comprises an administration amount.

3. The method of claim 1, wherein the dosing regimen comprises an administration schedule.

4. The method of claim 1, further comprising determining a size of the target tissue in the patient, wherein the created dosing regimen is further based on the size of the target tissue in the patient.

5. The method of claim 1, wherein the target tissue has been damaged, further comprising determining an extent of the damage, wherein the created dosing regimen is further based on the extent of the damage to the target tissue.

6. The method of claim 1, further comprising determining a weight or body composition of the patient, wherein the created dosing regimen is further based on the weight or body composition.

7. The method of claim 1, wherein the target tissue and the tissue sample are cartilage.

8. The method of claim 1, wherein the therapeutic protein is a fusion protein comprising a heparin binding (HB) peptide.

9. The method of claim 8, wherein the HB peptide is selected from the group consisting of KRKKKGKGLGKKRDPRLRKYK (SEQ ID NO:1) and KRKKKGKGLGKKRDPKLRKYK (SEQ ID NO:2)

10. The method of claim 8, wherein the fusion protein further comprises an active agent selected from the group consisting of a chemical entity to be administered to a subject to treat a condition and a biological product to be administered to a subject to treat a condition.

11. The method of claim 10, wherein the fusion protein further comprises a linker configured to couple the HB peptide to the active agent.

12. The method of claim 11, wherein the linker is a peptide comprising the sequence GGG.

13. The method of claim 1, wherein measuring the equilibrium uptake ratio comprises:
obtaining a radiolabeled version of the therapeutic protein;
incubating the tissue sample in a bath with the radiolabeled version of the therapeutic protein;
removing the tissue sample from the bath; and
measuring radioactivity in the removed tissue sample and the bath after the tissue sample is removed.

14. The method of claim 13, wherein the radiolabel comprises a radioisotope of iodine.

15. The method of claim 1, wherein determining the partition coefficient and determining the diffusivity comprises:
obtaining a radiolabeled version of the therapeutic protein;
incubating the tissue sample in a bath with the radiolabeled version of the therapeutic protein;
monitoring radioactivity of the bath during the incubating step;
determining a signal decay for the radioactivity of the bath during the incubating step; and
fitting the signal decay to a model to determine a product of the partition coefficient multiplied by the diffusivity.

16. The method of claim 13, wherein the radiolabel comprises a radioisotope of iodine.

17. A system for determining diffusivity of a compound into a tissue sample, the system comprising:
a bath comprising a radiolabeled compound;
a tissue sample located in the bath; and
a radiation detector positioned to detect radiation in the bath.

18. The system of claim 17 wherein the radiation detector is a radio-chromatography detector.

19. The system of claim 17, wherein the tissue sample is cartilage.

20. The system of claim 17, wherein the radiolabeled compound is a protein.

21. The system of claim 20, wherein the radiolabeled compound is a fusion protein comprising a heparin binding (HB) peptide.

22. The system of claim 21, wherein the HB peptide comprises a substitution at the cysteine of the naturally-occurring HB peptide.

22. The system of claim 21, wherein the fusion protein further comprises an active agent selected from the group consisting of a chemical entity to be administered to a subject to treat a condition and a biological product to be administered to a subject to treat a condition.

23. The system of claim 22, wherein the fusion protein further comprises a linker configured to couple the HB peptide to the active agent.

24. The system of claim 23, wherein the linker is a peptide comprising the sequence GGG.
